(19) United States
(12) Patent Application Publication
(10) Pub. No.: US 2025/0255921 A1
(43) Pub. Date: Aug. 14, 2025

(54) CHINESE HERBAL MEDICINE FORMULA AND ITS PREPARATION METHOD

(71) Applicant: Ning TSE, Hong Kong (HK)

(72) Inventors: Ning TSE, Hong Kong (HK); Yeungchu CHAN, Hong Kong (HK)

(21) Appl. No.: 19/196,640

(22) Filed: May 1, 2025

Related U.S. Application Data
(63) Continuation of application No. PCT/CN2023/131893, filed on Nov. 15, 2023.

(30) Foreign Application Priority Data
Nov. 17, 2022 (CN) 202211440550.6

(51) Int. Cl. A61K 36/738; A61K 33/04; A61K 33/243; A61K 36/284; A61K 36/285; A61K 36/286; A61K 36/287; A61K 36/346; A61K 36/532; A61K 36/534; A61K 36/535; A61K 36/61; A61K 36/634; A61K 36/736; A61K 36/752; A61K 36/758; A61K 36/82

(52) U.S. Cl. CPC A61K 36/738; A61K 33/04; A61K 33/243; A61K 36/284; A61K 36/285; A61K 36/286; A61K 36/287; A61K 36/346; A61K 36/532; A61K 36/534; A61K 36/535; A61K 36/61; A61K 36/634; A61K 36/736; A61K 36/752; A61K 36/758; A61K 36/82

(57) ABSTRACT

A Chinese herbal medicine formula and its preparation method are provided. The Chinese herbal medicine formula is made from the following raw materials in parts by weight: 8-12 parts of rose hydrosol, 1-3 parts of peppermint hydrosol, 28-42 parts of Chinese herbal medicine extract, 1-3 parts of chelated platinum ions, 1-3 parts of chelated selenium ions and 4-10 parts of composite antibacterial agent. The preparation method includes adding the rose hydrosol and the peppermint hydrosol to the Chinese herbal medicine extract to obtain a stock solution; diluting the stock solution to obtain a diluted solution; and adding the chelated platinum ions, the chelated selenium ions, and the composite antibacterial agent to the diluted solution, uniformly stirring to obtain the Chinese herbal medicine formula. The prepared Chinese herbal medicine formula can effectively kill bacteria, viruses, and fungi, has excellent killing effects, and will not cause harm to human body.

### CROSS-REFERENCE TO RELATED APPLICATIONS

This application is a continuation of International Patent Application No. PCT/CN2023/131893, filed Nov. 15, 2023. The International Patent Application claims priority to a Chinese patent application No. CN202211440550 filed on Nov. 17, 2022. The entire contents of the above-mentioned applications are hereby incorporated by reference.

### TECHNICAL FIELD

The disclosure relates to the field of disinfection technologies, and more particularly to a Chinese herbal medicine formula and its preparation method.

### BACKGROUND

With a development of society and an improvement of people's living standards, people pay more and more attention to harm of harmful microorganisms such as bacteria to people's health and living environment. Therefore, it is more and more important to actively explore long-lasting and effective antibacterial materials.

During use, Chinese herbal medicine disinfectants are not easy to produce drug resistance, and have characteristics of antibacterial, anti-inflammatory, low toxicity, low residue or no residue, and can effectively inhibit and kill pathogenic microorganisms. Reasons are that Chinese herbal medicine contains a variety of biologically active ingredients, which can interfere with a metabolism of pathogenic microorganisms in many links, such as energy conversion, DNA, and RNA. Chinese herbal medicine disinfectants are relatively simple in preparation process and equipment, widely available, low in cost and convenient to apply and promote.

Polymer antibacterial agents have a relatively high density of active functional groups, excellent antibacterial performance, and can kill bacteria through direct contact with bacteria without releasing active substances. As a result, their antibacterial performance is long-lasting and stable, with relatively low residual toxicity and high safety. According to different active functional groups, polymer antibacterial agents are mainly divided into quaternary ammonium salts, quaternary phosphonium salts, pyridine salts, halogenated amine, guanidine salts, chitosan, and so on. Among them, the quaternary ammonium salts are typical representatives of polymer antibacterial agents, and their characteristics are that a structural center is a positive quaternary ammonium nitrogen ion, surrounded by four substituents and counter anions. An antibacterial principle of quaternary ammonium salt polymer antibacterial agents is that a long-carbon-chain group connected to the quaternary ammonium nitrogen ion can pierce a lipid cell membrane of bacteria to achieve an effect of killing bacteria.

A Chinese Patent No. CN113413340A discloses a Chinese herbal medicine bacteriostatic agent and preparation method thereof, including the following components in parts by weight: 20-30 parts of Chinese herbal medicine extract, 0.8-1.2 parts of thickener, 0.5-1.5 parts of glycerol, 1-3 parts of sodium chloride, 0.03-0.08 parts of essence and 65-80 parts of pure water. The Chinese herbal medicine extract includes following components in parts by weight: 1-2 parts of cloves, 0.5-0.8 part of rhubarb, 0.2-0.6 part of Cinnamomum cassia, 0.8-1.2 part of Coptis root, 0.1-0.3 part of Lithospermum root, 0.2-0.5 part of Ephedra, 0.1-0.3 part of Ledum palustre, 35-45 parts of ethanol and 270-290 parts of pure water. Compared to the Chinese Patent No. CN113413340A, the disclosure uses rose hydrosol and peppermint hydrosol (also referred to as Mentha piperita hydrosol) as a base, while the Chinese Patent No. CN113413340A uses pure water. A hydrosol base of the disclosure has a unique efficacy and aroma. The disclosure adds chelated platinum ions and chelated selenium ions, which are not found in the Chinese Patent No. CN113413340A, and these two ions have special biological activities and synergistic effects. The disclosure uses a specially prepared composite antibacterial agent, which has stronger antibacterial effects and durability. Types and proportions of the Chinese herbal medicine used in the disclosure are significantly different from those in the Chinese Patent No. CN113413340A, which leads to different efficacies of final products.

A Chinese Patent No. CN111602658A discloses a degradable bactericide, including any one or both of a component A and a component B; the component A is a self-polymer obtained by self-polymerization of hydrophilic acrylate monomers, with a viscosity-average molecular weight in a range of 350-750; the component B is a random copolymer obtained by a copolymerization of hydrophilic acrylate monomers and hydrophobic acrylate monomers, with a viscosity-average molecular weight in a range of 600-900, a weight percentage of the hydrophilic acrylate monomers in the random copolymer is in a range of 20%- 35%. Compared to the Chinese Patent No. CN111602658A, in the disclosure, a bentonite is firstly subjected to sulfhydryl treatment, and subsequently dodecafluoroheptyl methacrylate and methacryloyloxyethyltrimethyl ammonium chloride are introduced into a surface of the sulfhydryl bentonite through polymerization reaction; and the bentonite is used as a basic material, so that an adsorption performance and immobilization performance of the composite antibacterial agent for effective ingredients in the Chinese herbal medicine extract are improved, thereby synergistically improving an antibacterial broad-spectrum and long-lasting performance of the composite antibacterial agent, while the Chinese Patent No. CN111602658A does not use the bentonite.

The disclosure aims to solve a problem of poor stability and insufficient activity of Chinese herbal medicine formula in related art, while the Chinese Patent No. CN113413340A and the Chinese Patent No. CN111602658A have not proposed or solved the problem. Because of different technical problems, it is difficult for those skilled in the art to get an inspiration from these two comparative documents to solve specific problems faced by the disclosure. The composite antibacterial agent of the disclosure adopts a unique preparation method, including sulfhydryl of bentonite and subsequent surface modification. The preparation method is fundamentally different from the preparation method of the bacteriostatic agent in the Chinese Patent No. CN113413340A. There is no hint in these two comparative documents that this special preparation method can be adopted. Therefore, those skilled in the art lack the inspiration to make such an innovation.

### SUMMARY

To solve deficiencies in related art, the disclosure aims to provide a Chinese herbal medicine formula and its preparation method, which have advantages of good product quality stability, readily available materials, low cost, excellent sterilization effect and no harm to human bodies.

To realize aforementioned objectives, the disclosure adopts following technical solutions.

The Chinese herbal medicine formula is made from following raw materials in parts by weight: 8-12 parts of rose hydrosol, 1-3 parts of peppermint hydrosol, 28-42 parts of Chinese herbal medicine extract, 1-3 parts of chelated platinum ions, 1-3 parts of chelated selenium ions and 4-10 parts of composite antibacterial agent.

The Chinese herbal medicine formula combines advantages of the Chinese herbal medicine extract, the chelating metal ions and the composite antibacterial agent, and has multiple synergistic antibacterial and anti-inflammatory effects. The Chinese herbal medicine extract contains a variety of biologically active ingredients, and can interfere with a metabolism of pathogenic microorganisms in many links. The chelated platinum ions and the chelated selenium ions enhance an antibacterial effect. The composite antibacterial agent provides long-lasting and stable antibacterial performance. This multiple action mechanism is less likely to produce drug resistance, and has characteristics of low toxicity and low residue. Further, an addition of the rose hydrosol and the peppermint hydrosol gives the Chinese herbal medicine formula a good aroma and enhances a use experience. Overall, the Chinese herbal medicine formula integrates advantages of natural plant extracts and modern synthetic materials so as to achieve efficient, safe and long-lasting antibacterial and disinfectant effects.

A preparation method of the composite antibacterial agent includes following two steps.

A sulfhydryl bentonite is prepared as follows. Firstly, a bentonite is calcined, cooled and pulverized to obtain a pulverized bentonite, which can increase its specific surface area. Then, the pulverized bentonite is dispersed into an ethanol aqueous solution to obtain a first mixed solution, and a potential of hydrogen (pH) of the first mixed solution is adjusted with hydrochloric acid to obtain an adjusted first mixed solution, which facilitates activating hydroxyl groups on a bentonite surface. Subsequently, 3-mercaptopropyltriethoxysilane is added into the adjusted first mixed solution to undergo a condensation reaction with the hydroxyl groups on the bentonite surface: Si—OH (bentonite surface)+(CH₃CH₂O)₃Si—CH₂CH₂CH₂—SH→Si—O—Si—CH₂CH₂CH₂—SH+3CH₃CH₂OH. The condensation reaction introduces sulfhydryl groups (—SH) on the bentonite surface to obtain a first product. Finally, the first product is centrifuged, washed and dried to obtain the sulfhydryl bentonite.

Step (1), calcining the bentonite to obtain a calcined bentonite, cooling the calcined bentonite to room temperature to obtain a cooled bentonite; pulverizing the cooled bentonite to obtain the pulverized bentonite, dispersing the pulverized bentonite into the ethanol aqueous solution to obtain the first mixed solution; adjusting the pH of the first mixed solution with hydrochloric acid to obtain the adjusted first mixed solution; adding 3-mercaptopropyl triethoxysilane into the adjusted first mixed solution to obtain a second mixed solution; then stirring the second mixed solution to undergo a reaction to obtain a first product; finally, centrifuging, washing, and drying the first product to obtain the sulfhydryl bentonite.

The composite antibacterial agent is prepared as follows. Firstly, monomers (dodecafluoroheptyl methacrylate and methacryloyloxyethyltrimethyl ammonium chloride) are dissolved in dimethylformamide (DMF) to obtain a mixture, and then the sulfhydryl bentonite is dispersed into the mixture to obtain a dispersed solution. A photoinitiator is added into the dispersed solution to undergo a reaction under an irradiation of ultraviolet (UV) light, and the UV light excites the photoinitiator to produce free radicals, thereby triggering a polymerization reaction of the monomers and reacting with the —SH groups on the bentonite surface to obtain sulfhydryl radicals: R·+HS-bentonite→RH+·S-bentonite, the sulfhydryl radicals participate in the polymerization reaction, so that a polymer chain is connected to the sulfhydryl bentonite through covalent bonds to obtain a second product: ·S-bentonite+n(monomer)→polymer-S-bentonite. Finally, the second product is centrifuged, washed and dried to obtain the composite antibacterial agent. In a preparation process of the composite antibacterial agent, fluoropolymer (to provide hydrophobicity) and quaternary ammonium salt polymer (to provide antibacterial activity) are grafted onto bentonite carrier with high specific surface area to form multifunctional composite antibacterial materials.

Step (2), adding dodecafluoroheptyl methacrylate, methacryloyloxyethyltrimethyl ammonium chloride into N,N-dimethylformamide and stirring well to obtain a mixture; then dispersing the sulfhydryl bentonite into the mixture, following by performing ultrasonic dispersion to obtain the dispersed solution; adding the photoinitiator into the dispersed solution to undergo the reaction under the irradiation of ultraviolet light to obtain the second product; finally, centrifuging the second product to obtain a centrifuged second product, washing the centrifuged second product with isopropanol to obtain a washed second product, and drying the washed second product to obtain the composite antibacterial agent.

In an embodiment, in the step (1), a concentration of the ethanol aqueous solution is in a range of 50 weight (wt) %-75wt %, and the pH of the first mixed solution is adjusted to a range of 3-5.

In an embodiment, in the step (1), a weight ratio of the bentonite to the ethanol aqueous solution and to the 3-mercaptopropyltriethoxysilane is in a range of 10:(30-50):(2-5).

In an embodiment, in the step (1), conditions of the calcining include: 150 CelsiusDegrees (° C.)-300° C. of calcination temperature, and 1 hour (h)-2 h of calcination time; conditions of stirring reaction include: nitrogen (N₂) as a stirring reaction atmosphere, 30° C.-45° C. of stirring reaction temperature, and 5 h-8 h of stirring reaction time.

In an embodiment, in the step (2), a weight ratio of the dodecafluoroheptyl methacrylate to the methacryloyloxyethyltrimethyl ammonium chloride to the N,N-dimethylformamide, and to the sulfhydryl bentonite is in a range of (4-7):(1-3):(30-50):10.

In an embodiment, in the step (2), the photoinitiator is one or more selected from a group consisting of 2,2-dimethoxy-2-phenylacetophenone, 2-ethoxy-1,2-diphenylethanone, and 2-butoxy-1,2-diphenylethanon.

In an embodiment, in the step (2), a time of the ultrasonic dispersion is in a range of 15 minutes (min)-30 min, a main wavelength of the ultraviolet light is in a range of 360 nanometers (nm)-365 nm, an intensity of the ultraviolet light is in a range of 45 milliwatts per square centimeter (mW/cm²)-75 mW/cm², a time of the irradiation is in a range of 60 min-90 min, and a temperature of the reaction is in a range of 35° C.-45° C.

The composite antibacterial agent grafts fluoropolymer and quaternary ammonium salt polymer onto the bentonite surface to obtain a novel composite material. This design possesses following benefits that: 1) the bentonite as a carrier provides a large specific surface area and increases a density of active groups; 2) quaternary ammonium salt groups can effectively pierce bacterial cell membrane; 3) fluoropolymer can provide excellent chemical stability and hydrophobicity; 4) antibacterial groups are immobilized by chemical bonding, which avoids a release of active substances and achieves long-lasting antibacterial effect. Additionally, the composite antibacterial agent further has good dispersibility and compatibility, and can be uniformly distributed in the Chinese herbal medicine formula. This composite design not only improves an antibacterial efficiency, but also prolongs an antibacterial duration and further reduces a potential impact on the environment.

In an embodiment, a preparation method of the Chinese herbal medicine extract includes following steps: weighing, in parts by weight, 1-2 parts of safflower, 8-12 parts of peach seed, 8-12 parts of forsythia fruit, 8-13 parts of chrysanthemum, 7-13 parts of perilla, 18-25 parts of Platycodon root, 8-11 parts of star anise, 9-14 parts of Aucklandia root, 7-12 parts of Agastache herba, 10-12 parts of Atractylodes rhizome, 10-12 parts of white Atractylodes, 9-13 parts of green tangerine peel, 10-12 parts of cloves, 4-6 parts of Sichuan pepper and 13-17 parts of tea leaves to obtain a Chinese herbal medicine mixture; and adding the Chinese herbal medicine mixture into a reaction vessel, then adding 500 parts of deionized water into the reaction vessel with the Chinese herbal medicine mixture to obtain a to-be-extracted Chinese herbal medicine mixture, and performing distillation extraction on the to-be-extracted Chinese herbal medicine mixture at a temperature in a range of 94° C.-97° C. for a time in a range of 1.5 h-3 h to obtain the Chinese herbal medicine extract.

The disclosure also claims a preparation method of the Chinese herbal medicine formula. The preparation method of the Chinese herbal medicine formula includes following steps: adding rose hydrosol and peppermint hydrosol into the Chinese herbal medicine extract to obtain a stock solution; diluting the stock solution to obtain a diluted stock solution, adding chelated platinum ions, chelated selenium ions, and the composite antibacterial agent into the diluted stock solution, and uniformly stirring to obtain the Chinese herbal medicine formula.

In an embodiment, a dilution factor of the stock solution is in a range of 10-20 times. Compared with the related art, the disclosure has following beneficial effects.

The safflower has effects of activating blood circulation and unblocking meridians; dissipating blood stasis and relieving pain. The safflower is used to treat amenorrhea, dysmenorrhea, lochiorrhea, chest impediment and angina, stagnant abdominal pain, hypochondriac stabbing pain, bruises and pain from sores.

The peach seed has effects of activating blood circulation and resolving stasis, moistening the intestines to relieve constipation, and suppressing cough and alleviating asthma. The peach seed is used to treat amenorrhea and dysmenorrhea, abdominal masses and stagnant lumps, pulmonary abscess and intestinal abscess, bruises, intestinal dryness and constipation, and cough and asthma.

The forsythia fruit has effects of clearing heat, detoxifying, dissipating nodules, and reducing swelling. The forsythia fruit is used to treat warm pathogen syndrome, erysipelas, skin rashes, carbuncles, scrofula, and dysuria.

The chrysanthemum has effects of dispersing wind-heat, clearing heat, brightening eyes and detoxifying. The chrysanthemum is used to treat headache, dizziness, red eyes, restlessness in the chest and heart, boils, and abscesses.

The perilla: leaves are diaphoretic, antitussive, and aromatic stomachic diuretics, have effects of analgesic, sedative, and detoxifying, and are used to treat colds; stems can regulate Qi and prevent miscarriage; seeds can suppress cough, eliminate phlegm, alleviate asthma, and relieve mental depression.

The Platycodon root has effects of diffusing lung Qi, soothing throat, eliminating phlegm and draining pus. The Platycodon root is used to treat excessive phlegm in cough, chest tightness and discomfort, sore throat, hoarseness, expectoration of pus in lung abscess, and and non-ulceration of pus in sores and ulcers.

The star anise is warm in nature and pungent in taste, has effects of warming yang to dispel cold and regulating Qi to relieve pain, and is used to treat cold-induced vomiting and hiccups, abdominal pain caused by cold hernia, kidney deficiency and lower back pain, and and dry or damp beriberi.

The Aucklandia root has effects of promoting Qi circulation to relieve pain and strengthening the spleen to aid digestion. The Aucklandia root is used to treat chest and epigastric distension and pain, tenesmus in diarrhea and dysentery, indigestion due to food retention, and loss of appetite.

The Agastache herba has effects of promoting Qi circulation, harmonizing the middle energizer, dispelling foulness, and removing dampness. The Agastache herba is used to treat summer-damp colds, chills and fever, headache, stuffiness in chest and epigastrium, vomiting and diarrhea, malaria, dysentery, and halitosis.

The Atractylodes rhizome has effects of strengthening the spleen, drying dampness, resolving depression, and dispelling foulness. The Atractylodes rhizome is used to treat spleen dysfunction caused by excessive dampness, fatigue and drowsiness, epigastric fullness and abdominal distension, loss of appetite, vomiting, diarrhea, dysentery, malaria, phlegm retention, edema, seasonal colds, wind-cold-damp arthralgia, foot weakness, and night blindness.

The white Atractylodes has effects of tonifying the spleen, benefiting the stomach, drying dampness, harmonizing the middle energizer, and preventing miscarriage. The white Atractylodes is used to treat weak spleen and stomach Qi, loss of appetite, fatigue with shortness of breath, abdominal distension, diarrhea, phlegm retention, edema, jaundice, dampness-induced arthralgia, difficulty in urination, dizziness, spontaneous sweating, and unstable fetal condition.

The green tangerine peel has effects of soothing the liver and breaking Qi stagnation, and eliminating food accumulation and resolving stagnation. The green tangerine is used to treat distending pain in the chest and hypochondrium, hernia, breast nodules, mastitis, and abdominal pain due to food accumulation.

The cloves have effects of warming the middle energizer, tonifying the kidneys, and descending rebellious Qi. The cloves are used to treat hiccups, vomiting, regurgitation, diarrhea and dysentery, cold pain in the heart and abdomen, abdominal masses, hernia, and skin diseases.

The Sichuan pepper has effects of warming the middle energizer and dispelling cold, removing dampness, relieving pain, killing parasites, and detoxifying fishy smells. The Sichuan pepper is used to treat food retention and fluid retention, cold pain in the heart and abdomen, vomiting, hiccups, cough with reversed Qi flow, arthralgia caused by wind-cold-dampness, diarrhea, dysentery, hernia pain, toothache, ascariasis, enterobiasis, pruritus vulvae, and scabies.

The tea leaves have effects of clearing the head and eyes, relieving irritability and thirst, resolving phlegm, aiding digestion, promoting diuresis, and detoxifying. The tea leaves are used to treat headache, blurred vision, excessive sleepiness, restlessness and thirst, food retention and phlegm stagnation, malaria, and dysentery.

The disclosure discloses a Chinese herbal medicine formula; using plant ingredients and composite antibacterial agent with effects of anti-inflammation, anti-oxidation, anti-allergy, reducing infection and enhancing immunity, as well as chelated platinum ions and chelated selenium ions that play a catalytic role in enhancing a performance of Chinese herbal medicine as raw materials, a formulated nursing solution has excellent bactericidal and antibacterial effects, can further effectively moisturize and nourish human skin, and can be used in a spraying way, which is convenient and fast.

The disclosure provides a composite antibacterial agent; firstly, the bentonite is organically modified with 3-mercaptopropyltriethoxysilane to obtain the sulfhydryl bentonite, and the sulfhydryl bentonite can effectively improve a dispersibility of the bentonite in the Chinese herbal medicine extract and prevent the bentonite from aggregation and sedimentation in a medicine. The dodecafluoroheptyl methacrylate and the methacryloyloxyethyltrimethyl ammonium chloride are grafted onto the sulfhydryl bentonite by a mercaptoene reaction under the irradiation of the ultraviolet light to obtain the composite antibacterial agent. The methacryloyloxyethyltrimethyl ammonium chloride can change a membrane permeability of bacterial cells, causing the bacterial cells to die; further has good surface activity and can accumulate on surfaces of the bacterial cells to affect bacterial metabolism; additionally can denature proteins and inactivate a bacterial enzyme system; is safe and non-toxic to human body, non-irritating to skin, and will not cause drug resistance to bacteria, so it can bring excellent antibacterial activity. The dodecafluoroheptyl methacrylate has excellent hydrophobicity, which can prevent an initial adhesion between bacteria and protein to inhibit a formation of biofilm, therefore working synergistically with methacryloyloxyethyltrimethyl ammonium chloride to suppress and kill bacteria.

### DETAILED DESCRIPTION OF EMBODIMENTS

To make the objectives, technical solutions and advantages of the disclosure clearer, the disclosure will be further described in detail with reference to embodiments. It should be noted that, the specific embodiments described here are only used to explain the disclosure, and are not used to limit the disclosure.

Unless otherwise indicated, chemical reagents and materials in the disclosure are purchased by market means or synthesized from raw materials purchased by market means.

Bentonite is purchased from Haoyuan Bentonite Factory (Fangzi District, Weifang City, China), with a particle size of 4,000-5,000 mesh.

Both rose hydrosol and peppermint hydrosol are purchased from Guangzhou Zhonghe International Biotechnology Co., Ltd.

The chelated platinum ions and chelated selenium ions are purchased from Yatian Technology Group.

The disclosure will be further described with reference to specific embodiments.

### Embodiment 1

A preparation method of a Chinese herbal medicine formula includes following four steps.

Step (1), 10 grams (g) of bentonite is calcined at a temperature of 300° C. for 2 h to obtain a calcined bentonite, the calcined bentonite is cooled to room temperature to obtain a cooled bentonite, then the cooled bentonite is pulverized to obtain a pulverized bentonite. The pulverized bentonite is dispersed into 50 g of 70wt % ethanol aqueous solution to obtain a first mixed solution, and a pH of the first mixed solution is adjusted to 5 with hydrochloric acid to obtain an adjusted first mixed solution. Then, 5 g of 3-mercaptopropyltricthoxysilane is added into the adjusted first mixed solution to obtain a second mixed solution, the second mixed solution is stirred to undergo a reaction at 45° C. in a nitrogen (N₂) atmosphere for 8 h to obtain a first product, and the first product is centrifuged, washed, and dried to obtain a sulfhydryl bentonite.

Step (2), 7 g of dodecafluoroheptyl methacrylate and 3 g of methacryloyloxyethyltrimethyl ammonium chloride are added into 50 g of N,N-dimethylformamide and stirred well to obtain a mixture. Then, 10 g of the sulfhydryl bentonite is dispersed into the mixture following by performing ultrasonic dispersion for 30 min to obtain a dispersed solution; then 2-butoxy-1,2-diphenylethanone is added into the dispersed solution to undergo a reaction for 90 min under an irradiation of ultraviolet light with a main wavelength of 365 nm and an intensity of 60 mW/cm² at 45° C. to obtain a second product. Finally, the second product is centrifuged to obtain a centrifuged second product, the centrifuged second product is washed with isopropanol to obtain a washed second product, and the washed second product is dried to obtain a composite antibacterial agent.

Step (3), 2 g of safflower, 10 g of peach seed, 10 g of forsythia fruit, 11 g of chrysanthemum, 10 g of perilla, 22 g of Platycodon root, 9 g of star anise, 12 g of Aucklandia root, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture; the Chinese herbal medicine mixture is added into a reaction vessel. Then, 500 g of deionized water is added into a reaction vessel with the Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (4), 12 g of rose hydrosol and 3 g of peppermint hydrosol are added into 42 g of the Chinese herbal medicine extract to obtain a stock solution; the stock solution is diluted 20 times to obtain a diluted solution, 3 g of chelated platinum ions, 3 g of chelated selenium ions, and 10 g of the composite antibacterial agent are added into the diluted solution, and uniformly stirred to obtain the Chinese herbal medicine formula (i.e., a Chinese herbal medicine composition).

### Embodiment 2

A preparation method of a Chinese herbal medicine formula includes following four steps.

Step (1), 10 g of bentonite is calcined at a temperature of 200° C. for 1.5 h to obtain a calcined bentonite, the calcined bentonite is cooled to room temperature to obtain a cooled bentonite, then the cooled bentonite is pulverized to obtain a pulverized bentonite. The pulverized bentonite is dispersed into 40 g of 70 wt % ethanol aqueous solution to obtain a first mixed solution; and a pH of the first mixed solution is adjusted to 4 with the hydrochloric acid to obtain an adjusted first mixed solution. Then 4 g of the 3-mercaptopropyltriethoxysilane is added into the adjusted first mixed solution to obtain a second mixed solution, the second mixed solution is stirred to undergo reaction at 35° C. in the N2 atmosphere for 7 h to obtain a first product, and the first product is centrifuged, washed, and dried to obtain a sulfhydryl bentonite.

Step (2), 5 g of the dodecafluoroheptyl methacrylate and 2 g of the methacryloyloxyethyl trimethyl ammonium chloride are added into 40 g of the N,N-dimethylformamide and stirred well to obtain a mixture. Then 10 g of the sulfhydryl bentonite is dispersed into the mixture following by performing ultrasonic dispersion for 20 min to obtain a dispersed solution; then the 2-butoxy-1,2-diphenylethanone is added into the dispersed solution to undergo a reaction for 75 min under the irradiation of ultraviolet light with the main wavelength of 365 nm and the intensity of 60 mW/cm² at 40° C. to obtain a second product. Finally, the second product is centrifuged to obtain a centrifuged second product; the centrifuged second product is washed with isopropanol to obtain a washed second product; and the washed second product is dried to obtain a composite antibacterial agent.

Step (3), 2 g of safflower, 10 g of peach seed, 10 g of forsythia fruit, 11 g of chrysanthemum, 10 g of perilla, 22 g of Platycodon root, 9 g of star anise, 12 g of Aucklandia root, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture; the Chinese herbal medicine mixture is added into a reaction vessel. Then 500 g of deionized water is added into a reaction vessel with Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (4), 10 g of rose hydrosol and 2 g of peppermint hydrosol are added into 35 g of the Chinese herbal medicine extract to obtain a stock solution; the stock solution is diluted 20 times to obtain a diluted solution, 2 g of chelated platinum ions, 2 g of chelated selenium ions, and 7 g of the composite antibacterial agent are added into the diluted solution, and uniformly stirred to obtain the Chinese herbal medicine formula.

### Embodiment 3

A preparation method of a Chinese herbal medicine formula, includes following four steps.

Step (1), 10 g of bentonite is calcined at a temperature of 150° C. for 1 h to obtain a calcined bentonite, the calcined bentonite is cooled to room temperature to obtain a cooled bentonite, then the cooled bentonite is pulverized to obtain a pulverized bentonite. The pulverized bentonite is dispersed into 30 g of 70wt % ethanol aqueous solution to obtain a first mixed solution; and a pH of the first mixed solution is adjusted to 3 with hydrochloric acid to obtain an adjusted first mixed solution. Then 2 g of 3-mercaptopropyl triethoxysilane is added into the adjusted first mixed solution to obtain a second mixed solution, the second mixed solution is stirred to undergo reaction at 30° C. in the N₂ atmosphere for 5 h to obtain a first product, and the first product is centrifuged, washed, and dried to obtain a sulfhydryl bentonite.

Step (2), 4 g of dodecafluoroheptyl methacrylate and 1 g of methacryloyloxyethyl trimethyl ammonium chloride are added into 30 g of N,N-dimethylformamide and stirred well to obtain a mixture. Then 10 g of the sulfhydryl bentonite is dispersed into the mixture following by performing ultrasonic dispersion for 15 min to obtain a dispersed solution; then 2-butoxy-1,2-diphenylethanone is added into the dispersed solution to undergo a reaction for 60 min under the irradiation of ultraviolet light with the main wavelength of 365 nm and the intensity of 60 mW/cm² at 35° C. to obtain a second product. Finally, the second product is centrifuged to obtain a centrifuged second product; the centrifuged second product is washed with isopropanol to obtain a washed second product; and the washed second product is dried to obtain a composite antibacterial agent.

Step (3), 2 g of safflower, 10 g of peach seed, 10 g of forsythia fruit, 11 g of chrysanthemum, 10 g of perilla, 22 g of Platycodon root, 9 g of star anise, 12 g of Aucklandiaroot, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture; the Chinese herbal medicine mixture is added into a reaction vessel. Then 500 g of deionized water is added into a reaction vessel with the Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (4), 8 g of rose hydrosol and 1 g of peppermint hydrosol are added into 28 g of the Chinese herbal medicine extract to obtain a stock solution; the stock solution is diluted 20 times to obtain a diluted stock solution, 1 g of chelated platinum ions, 1 g of chelated selenium ions, and 4 g of the composite antibacterial agent are added into the diluted stock solution, and uniformly stirred to obtain the Chinese herbal medicine formula.

### Comparative Embodiment 1

A preparation method of a Chinese herbal medicine formula, includes following four steps.

Step (1), 10 g of bentonite is calcined at a temperature of 300° C. for 2 h to obtain a calcined bentonite, the calcined bentonite is cooled to room temperature to obtain a cooled bentonite, then the cooled bentonite is pulverized to obtain a pulverized bentonite. The pulverized bentonite is dispersed into 50 g of 70 wt % ethanol aqueous solution to obtain a first mixed solution, and a pH of the first mixed solution is adjusted to 5 with hydrochloric acid to obtain an adjusted first mixed solution. Then 5 g of 3-mercaptopropyltriethoxysilane is added into the adjusted first mixed solution to obtain a second mixed solution, the second mixed solution is stirred to undergo a reaction at 45° C. in the N₂ atmosphere for 8 h to obtain a first product, and the first product is centrifuged, washed, and dried to obtain a sulfhydryl bentonite.

Step (2), 7 g of dodecafluoroheptyl methacrylate is added into 50 g of N,N-dimethylformamide and stirred well to obtain a mixture. Then 10 g of the sulfhydryl bentonite is dispersed into the mixture following by performing ultrasonic dispersion for 30 min to obtain a dispersed solution; then 2-butoxy-1,2-diphenylethanone is added into the dispersed solution to undergo a reaction for 90 min under the irradiation of ultraviolet light with the main wavelength of 365 nm and the intensity of 60 mW/cm² at 45° C. to obtain a second product. Finally, the second product is centrifuged to obtain a centrifuged second product; the centrifuged second product is washed with isopropanol to obtain a washed second product; and the washed second product is dried to obtain an antibacterial agent.

Step (3), 2 g of safflower, 10 g of peach seed, 10 g of forsythia fruit, 11 g of chrysanthemum, 10 g of perilla, 22 g of Platycodon root, 9 g of star anise, 12 g of Aucklandiaroot, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture; the Chinese herbal medicine mixture is added into a reaction vessel. Then 500 g of deionized water is added into a reaction vessel with the Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (4), 12 g of rose hydrosol and 3 g of peppermint hydrosol are added into 42 g of the Chinese herbal medicine extract to obtain a stock solution; the stock solution is diluted 20 times to obtain a diluted stock solution, 3 g of chelated platinum ions, 3 g of chelated selenium ions, and 10 g of the antibacterial agent are added into the diluted stock solution, and uniformly stirred to obtain the Chinese herbal medicine formula.

### Comparative Embodiment 2

A preparation method of a Chinese herbal medicine formula, includes following four steps.

Step (1), 10 g of bentonite is calcined at a temperature of 300° C. for 2 h to obtain a calcined bentonite, the calcined bentonite is cooled to room temperature to obtain a cooled bentonite, then the cooled bentonite is pulverized to obtain a pulverized bentonite. The pulverized bentonite is dispersed to 50 g of 70wt % ethanol aqueous solution to obtain a first mixed solution, and a pH of the first mixed solution is adjusted to 5 with hydrochloric acid to obtain an adjusted first mixed solution. Then 5 g of 3-mercaptopropyltriethoxysilane is added into the adjusted first mixed solution to obtain a second mixed solution, the second mixed solution is stirred to undergo reaction at 45° C. in the N2 atmosphere for 8 h to obtain a first product, and the first product is centrifuged, washed, and dried to obtain sulfhydryl bentonite.

Step (2), 3 g of methacryloyloxyethyl trimethyl ammonium chloride is added into 50 g of N,N-dimethylformamide and stirred well to obtain a mixture. Then 10 g of the sulfhydryl bentonite is dispersed into the mixture following by performing ultrasonic dispersion for 30 min to obtain a dispersed solution; then 2-butoxy-1,2-diphenylethanone is added into the dispersed solution to undergo a reaction for 90 min under the irradiation of ultraviolet light with the main wavelength of 365 nm and the intensity of 60 mW/cm² at 45° C. to obtain a second product. Finally, the second product is centrifuged to obtain a centrifuged second product; the centrifuged second product is washed with isopropanol to obtain a washed second product; and the washed second product is dried to obtain an antibacterial agent.

Step (3), 2 g of safflower, 10 g of peach seed, 10 g of forsythia fruit, 11 g of chrysanthemum, 10 g of perilla, 22 g of Platycodon root, 9 g of star anise, 12 g of Aucklandia root, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture, the Chinese herbal medicine mixture is added into a reaction vessel. Then 500 g of deionized water is added into a reaction vessel with the Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (4), 12 g of rose hydrosol and 3 g of peppermint hydrosol are added into 42 g of the Chinese herbal medicine extract to obtain a stock solution; the stock solution is diluted 20 times to obtain a diluted stock solution, 3 g of chelated platinum ions, 3 g of chelated selenium ions, and 10 g of the antibacterial agent are added into the diluted solution, and uniformly stirred to obtain the Chinese herbal medicine formula.

### Comparative Embodiment 3

A preparation method of a Chinese herbal medicine formula, includes following three steps.

Step (1), 10 g of bentonite is calcined at a temperature of 300° C. for 2 h to obtain a calcined bentonite, the calcined bentonite is cooled to room temperature to obtain a cooled bentonite, then the cooled bentonite is pulverized to obtain a pulverized bentonite. The pulverized bentonite is dispersed to 50 g of 70wt % ethanol aqueous solution to obtain a first mixed solution, and a pH of the first mixed solution is adjusted to 5 with hydrochloric acid to obtain an adjusted first mixed solution. Then 5 g of 3-mercaptopropyltriethoxysilane is added into the adjusted first mixed solution to obtain a second mixed solution, the second mixed solution is stirred and reacted at 45° C. in the N2 atmosphere for 8 h to obtain a first product, and the first product is centrifuged, washed, and dried to obtain a sulfhydryl bentonite.

Step (2), 2 g of safflower, 10 g of peach seed, 10 g of forsythia fruit, 11 g of chrysanthemum, 10 g of perilla, 22 g of Platycodon root, 9 g of star anise, 12 g of Aucklandiaroot, 8 g of Agastache herba, 11 g of Atractylodes rhizome, 11 g of white Atractylodes, 11 g of green tangerine peel, 11 g of cloves, 5 g of Sichuan pepper and 16 g of tea leaves are weighed to obtain a Chinese herbal medicine mixture; the Chinese herbal medicine mixture is added into a reaction vessel. Then 500 g of deionized water is added into a reaction vessel with the Chinese herbal medicine mixture to obtain a to-be-extracted Chinese herbal medicine mixture, and the to-be-extracted Chinese herbal medicine mixture is performed with distillation extraction at 96° C. for 2.5 h to obtain a Chinese herbal medicine extract.

Step (3), 12 g of rose hydrosol and 3 g of peppermint hydrosol are added into 42 g of the Chinese herbal medicine extract to obtain a stock solution; the stock solution is diluted 20 times to obtain a diluted stock solution, 3 g of chelated platinum ions, 3 g of chelated selenium ions, and 10 g of the sulfhydryl bentonite are added into the diluted stock solution, and uniformly stirred to obtain the Chinese herbal medicine formula.

Escherichia coli and Staphylococcus aureus are selected as experimental strains, and are activated in a broth medium at 37° C. for 8 hours with a shaker rotation speed of 150 revolutions per minute (rpm), and then are ready for use. Chinese herbal medicine formula products prepared in embodiments 1-3 and comparative embodiments 1-3, which have been sterilized at 121° C. for 20 minutes, are respectively added to a sterile broth medium at a concentration of 0.05% (weight/volume (w/v)). Under sterile conditions, 1 milliliter (mL) of activated bacterial solution (with a bacterial concentration of 109 colony-forming units per milliliter abbreviated as CFU/mL) is taken and added to 49 mL of mediums prepared as described above, and then is cultured at 37°° C. for 6 h with a shaker rotation speed of 150 rpm to obtain a sample. After that, according to GB 4789.2-2022 “National Food Safety Standard-Microbiological Examination of Food: Enumeration of Total Bacterial Colonies”, samples after diluting are characterized by a plate count method. A bactericidal rate is calculated using following formula: Bactericidal Rate (%)=(Control Group Bacterial Count-Experimental Group Bacterial Count)/Control Group Bacterial Count×100, where bacterial counts are expressed in CFU/mL. The test results are shown in Table 1.

TABLE 1
Bactericidal Rate of Chinese herbal medicine formulas

| | 1 min | | 5 min | | 10 min | |
|---|---|---|---|---|---|---|
| | Escherichia | Staphylococcus | Escherichia | Staphylococcus | Escherichia | Staphylococcus |
| | coli | aureus | coli | aureus | coli | aureus |
| Embodiment 1 | 99.67 | 83.54 | 99.99 | 87.62 | 99.99 | 94.65 |
| Embodiment 2 | 99.52 | 82.39 | 99.99 | 86.24 | 99.99 | 93.97 |
| Embodiment 3 | 99.46 | 82.15 | 99.99 | 85.19 | 99.99 | 92.83 |
| Comparative | 76.71 | 61.32 | 81.27 | 64.53 | 83.64 | 69.48 |
| Embodiment 1 | | | | | | |
| Comparative | 86.36 | 70.26 | 88.59 | 75.37 | 90.15 | 81.54 |
| Embodiment 2 | | | | | | |
| Comparative | 72.93 | 52.91 | 76.52 | 59.46 | 81.52 | 65.32 |
| Embodiment 3 | | | | | | |

Aforementioned descriptions are merely part of embodiments of the disclosure, but a scope of protection of the disclosure is not limited thereto. Within a technical scope disclosed by the disclosure, equivalent substitutions or alterations based on the technical solutions and inventive concept of the disclosure made by those skilled in the art fall within the scope of protection of the disclosure.

What is claimed is:

1. A Chinese herbal medicine formula, wherein the Chinese herbal medicine formula is made from following raw materials in parts by weight: 8-12 parts of rose hydrosol, 1-3 parts of peppermint hydrosol, 28-42 parts of Chinese herbal medicine extract, 1-3 parts of chelated platinum ions, 1-3 parts of chelated selenium ions and 4-10 parts of composite antibacterial agent;
wherein a preparation method of the composite antibacterial agent comprises following steps:
step (1), calcining a bentonite to obtain a calcined bentonite; cooling the calcined bentonite to room temperature to obtain a cooled bentonite; pulverizing the cooled bentonite to obtain a pulverized bentonite; and dispersing the pulverized bentonite into an ethanol aqueous solution to obtain a first mixed solution; adjusting a potential of hydrogen (pH) of the first mixed solution with hydrochloric acid to obtain an adjusted first mixed solution; adding 3-mercaptopropyltriethoxysilane into the adjusted first mixed solution to obtain a second mixed solution; stirring the second mixed solution to undergo a reaction to obtain a first product; and centrifuging, washing, and drying the first product to obtain a sulfhydryl bentonite; and
step (2), adding dodecafluoroheptyl methacrylate and methacryloyloxyethyltrimethyl ammonium chloride into N,N-dimethylformamide and stirring well to obtain a mixture; then dispersing the sulfhydryl bentonite into the mixture, following by performing ultrasonic dispersion to obtain a dispersed solution; then adding a photoinitiator into the dispersed solution to undergo a reaction under an irradiation of a ultraviolet light to obtain a second product; finally, centrifuging the second product to obtain a centrifuged second product; washing the centrifuged second product with isopropanol to obtain a washed second product; and drying the washed second product to obtain the composite antibacterial agent.

2. The Chinese herbal medicine formula as claimed in claim 1, wherein, in the step (1), a concentration of the ethanol aqueous solution is in a range of 50 weight (wt) %- 75 wt %, and the pH of the first mixed solution is adjusted to a range of 3-5.

3. The Chinese herbal medicine formula as claimed in claim 1, wherein, in the step (1), a weight ratio of the bentonite to the ethanol aqueous solution and to the 3-mercaptopropyltriethoxysilane is in a range of 10:(30-50):(2-5).

4. The Chinese herbal medicine formula as claimed in claim 1, wherein, in the step (1), conditions of the calcining comprise: 150 Celsius degrees (° C.)-300° C. of calcination temperature and 1 hour (h)-2 h of calcination time; reaction conditions of the stirring comprise nitrogen (N2) as a stirring reaction atmosphere, 30° C.-45° C. of stirring reaction temperature, and 5 h-8 h of stirring reaction time.

5. The Chinese herbal medicine formula as claimed in claim 1, wherein, in the step (2), a weight ratio of the dodecafluoroheptyl methacrylate to the methacryloyloxyethyltrimethylammonium chloride to the N,N-dimethylformamide, and to the sulfhydryl bentonite is in a range of (4-7):(1-3):(30-50):10.

6. The Chinese herbal medicine formula as claimed in claim 1, wherein, in the step (2), the photoinitiator is one or more selected from the group consisting of 2,2-dimethoxy-2-phenylacetophenone, 2-ethoxy-1,2-diphenylethanone, and 2-butoxy-1,2-diphenylethanone.

7. The Chinese herbal medicine formula as claimed in claim 1, wherein, in the step (2), a time of the ultrasonic dispersion is in a range of 15 minutes (min)-30 min, a main wavelength of the ultraviolet light is in a range of 360 nanometers (nm)-365 nm, an intensity of the ultraviolet light is in a range of 45 milliwatts per square centimeter (mW/cm²)-75 mW/cm², a time of the irradiation is in a range of 60 min-90 min, and a temperature of the reaction is in a range of 35° C.-45° C.

8. The Chinese herbal medicine formula as claimed in claim 1, wherein a preparation method of the Chinese herbal medicine extract comprises following steps: weighing, in parts by weight, 1-2 parts of safflower, 8-12 parts of peach seed, 8-12 parts of forsythia fruit, 8-13 parts of chrysanthemum, 7-13 parts of perilla, 18-25 parts of Platycodon root, 8-11 parts of star anise, 9-14 parts of Aucklandia root, 7-12 parts of Agastache herba, 10-12 parts of Atractylodesrhizome, 10-12 parts of white Atractylodes, 9-13 parts of green tangerine peel, 10-12 parts of cloves, 4-6 parts of Sichuan pepper and 13-17 parts of tea leaves to obtain a Chinese herbal medicine mixture; adding the Chinese herbal medicine mixture into a reaction vessel; then adding 500 parts of deionized water into the reaction vessel with the Chinese herbal medicine mixture to obtain a to-be-extracted Chinese herbal medicine mixture; and performing distillation extraction on the to-be-extracted Chinese herbal medicine mixture at a temperature in a range of 94° C.-97° C. for a time in a range of 1.5 h-3 h to obtain the Chinese herbal medicine extract.

9. A preparation method of a Chinese herbal medicine formula, comprising following steps:
adding, in parts by weight, 8-12 parts of rose hydrosol and 1-3 parts of peppermint hydrosol into 28-42 parts of Chinese herbal medicine extract to obtain a stock solution; diluting the stock solution to obtain a diluted stock solution; adding 1-3 parts of chelated platinum ions, 1-3 parts of chelated selenium ions, and 4-10 parts of composite antibacterial agent into the diluted stock solution, and uniformly stirring to obtain the Chinese herbal medicine formula;
wherein the composite antibacterial agent is prepared by following steps:
step (1), calcining a bentonite to obtain a calcined bentonite, cooling the calcined bentonite to room temperature to obtain a cooled bentonite, pulverizing the cooled bentonite to obtain a pulverized bentonite, and dispersing the pulverized bentonite into an ethanol aqueous solution to obtain a first mixed solution; adjusting a pH of the first mixed solution with hydrochloric acid to obtain an adjusted first mixed solution; adding 3-mercaptopropyltriethoxysilane into the adjusted first mixed solution to obtain a second mixed solution; stirring the second mixed solution to undergo a reaction to obtain a first product; and centrifuging, washing, and drying the first product to obtain a sulfhydryl bentonite; and
step (2), adding dodecafluoroheptyl methacrylate and methacryloyloxyethyltrimethyl ammonium chloride into N,N-dimethylformamide and stirring well to obtain a mixture; then dispersing the sulfhydryl bentonite into the mixture, following by performing ultrasonic dispersion to obtain a dispersed solution; then adding a photoinitiator into the dispersed solution to undergo a reaction under an irradiation of ultraviolet light to obtain a second product; finally, centrifuging the second product to obtain a centrifuged second product, washing the centrifuged second product with isopropanol to obtain a washed second product, and drying the washed second product to obtain the composite antibacterial agent.

10. The preparation method of the Chinese herbal medicine formula as claimed in claim 9, wherein, in the step (1), a concentration of the ethanol aqueous solution is in a range of 50 wt %- 75wt %, and the pH of the first mixed solution is adjusted to a range of 3-5.

11. The preparation method of the Chinese herbal medicine formula as claimed in claim 9, wherein, in the step (1), a weight ratio of the bentonite to the ethanol aqueous solution and to the 3-mercaptopropyltriethoxysilane is in a range of 10:(30-50):(2-5).

12. The preparation method of the Chinese herbal medicine formula as claimed in claim 9, wherein, in the step (1), conditions of the calcining comprise: 150° C.-300° C. of calcination temperature, and 1 h-2 h of calcination time; reaction conditions of the stirring comprise: N₂ as a stirring reaction atmosphere, 30° C.-45° C. of stirring reaction temperature, and 5 h-8 h of stirring reaction time.

13. The preparation method of the Chinese herbal medicine formula as claimed in claim 9, wherein, in the step (2), a weight ratio of the dodecafluoroheptyl methacrylate to the methacryloyloxyethyltrimethyl ammonium chloride to the N,N-dimethylformamide, and to the sulfhydryl bentonite is in a range of (4-7):(1-3):(30-50):10.

14. The preparation method of the Chinese herbal medicine formula as claimed in claim 9, wherein, in the step (2), the photoinitiator is one or more selected from the group consisting of 2,2-dimethoxy-2-phenylacetophenone, 2-ethoxy-1,2-diphenylethanone, and 2-butoxy-1,2-diphenylethanone.

15. The preparation method of the Chinese herbal medicine formula as claimed in claim 9, wherein, in the step (2), a time of ultrasonic dispersion is in a range of 15 min-30 min, a main wavelength of the ultraviolet light is in a range of 360 nm-365 nm, an intensity of the ultraviolet light is in a range of 45 mW/cm²-75 mW/cm², a time of the irradiation is in a range of 60 min-90 min, and a temperature of the reaction is in a range of 35° C.-45° C.

16. The preparation method of the Chinese herbal medicine formula as claimed in claim 9, wherein a preparation method of the Chinese herbal medicine extract comprises following steps: weighing, in parts by weight, 1-2 parts of safflower, 8-12 parts of peach seed, 8-12 parts of forsythia fruit, 8-13 parts of chrysanthemum, 7-13 parts of perilla, 18-25 parts of Platycodonroot, 8-11 parts of star anise, 9-14 parts of Aucklandia root, 7-12 parts of Agastache herba, 10-12 parts of Atractylodes rhizome, 10-12 parts of white Atractylodes, 9-13 parts of green tangerine peel, 10-12 parts of cloves, 4-6 parts of Sichuan pepper and 13-17 parts of tea leaves to obtain a Chinese herbal medicine mixture; adding the Chinese herbal medicine mixture into a reaction vessel; then adding 500 parts of deionized water into the reaction vessel with the Chinese herbal medicine mixture to obtain a to-be-extracted Chinese herbal medicine mixture; and performing distillation extraction on the to-be-extracted Chinese herbal medicine mixture at a temperature in a range of 94° C.-97° C. for a time in a range of 1.5 h-3 h to obtain the Chinese herbal medicine extract.

17. The preparation method of the Chinese herbal medicine formula as claimed in claim 9, wherein a dilution factor of the stock solution is in a range of 10-20 times.

18. A Chinese herbal medicine composition, wherein the Chinese herbal medicine composition is made from following raw materials: a rose hydrosol, a peppermint hydrosol, a Chinese herbal medicine extract, chelated platinum ions, chelated selenium ions, and a composite antibacterial agent.

19. The Chinese herbal medicine composition as claimed in claim 18, wherein the composite antibacterial agent is prepared by following steps:
(1) calcining 10 g of bentonite at a temperature of 300° C. for 2 h to obtain a calcined bentonite; cooling the calcined bentonite to room temperature to obtain a cooled bentonite; pulverizing the cooled bentonite to obtain a pulverized bentonite; and dispersing the pulverized bentonite into 50 g of 70wt % ethanol aqueous solution to obtain a first mixed solution; adjusting a pH of the first mixed solution to 5 with hydrochloric acid to obtain an adjusted first mixed solution; adding 5 g of 3-mercaptopropyltriethoxysilane into the adjusted first mixed solution to obtain a second mixed solution; stirring the second mixed solution to undergo a reaction at 45° C. in a N₂ atmosphere for 8 h to obtain a first product; and centrifuging, washing, and drying the first product to obtain a sulfhydryl bentonite; and
(2) adding 7 g of dodecafluoroheptyl methacrylate and 3 g of methacryloyloxyethyl trimethyl ammonium chloride into 50 g of N,N-dimethylformamide and stirring well to obtain a mixture; then dispersing 10 g of the sulfhydryl bentonite into the mixture, following by performing ultrasonic dispersion for 30 min to obtain a dispersed solution; then adding 2-butoxy-1,2-diphenylethanone into the dispersed solution to undergo a reaction for 90 min under an irradiation of ultraviolet light with a main wavelength of 365 nm and an intensity of 60 mW/cm2 at a temperature of 45° C. to obtain a second product; finally, centrifuging the second product to obtain a centrifuged second product, washing the centrifuged second product with isopropanol to obtain a washed second product, and drying the washed second product to obtain the composite antibacterial agent.

20. The Chinese herbal medicine composition as claimed in claim 19, wherein the Chinese herbal medicine extract is prepared by following steps: weighing 2 g of safflower, 10 g of peach seed, 10 g of forsythia fruit, 11 g of chrysanthemum, 10 g of perilla, 22 g of Platycodon root, 9 g of star anise, 12 g of Aucklandia root, 8 g of Agastache herba, 11 g of Atractylodes rhizome, 11 g of white Atractylodes, 11 g of green tangerine peel, 11 g of cloves, 5 g of Sichuan pepper and 16 g of tea leaves to obtain a Chinese herbal medicine mixture; adding the Chinese herbal medicine mixture into a reaction vessel; then adding 500 g of deionized water into the reaction vessel with the Chinese herbal medicine mixture to obtain a to-be-extracted Chinese herbal medicine mixture; and performing distillation extraction on the to-be-extracted Chinese herbal medicine mixture at a temperature of 96° C. for 2.5 h to obtain the Chinese herbal medicine extract.
